(19) United States
(12) Patent Application Publication
(10) Pub. No.: US 2024/0008913 A1
(43) Pub. Date: Jan. 11, 2024

(54) METHOD AND SYSTEM FOR DETECTING LIQUID IMMERSION OF AN END EFFECTOR OF AN ULTRASONIC INSTRUMENT

(71) Applicant: Verb Surgical Inc., Santa Clara, CA (US)

(72) Inventors: Amirhossein Farvardin, San Jose, CA (US); Berk Gonenc, San Jose, CA (US); Guion Y. Lucas, Cincinnati, OH (US)

(21) Appl. No.: 17/862,328

(22) Filed: Jul. 11, 2022

(51) Int. Cl. A61B 18/14; A61B 17/32

(52) U.S. Cl. CPC A61B 18/1445; A61B 17/320092; A61B 2017/00026

(57) ABSTRACT

A method performed by a surgical system. The method determines one or more characteristics of an end effector of an ultrasonic instrument and determines that the end effector is at least partially submerged within a liquid based on the determined one or more characteristics. In response, the method displays a notification on a display of the surgical system indicating that the end effector is at least partially submerged within the liquid.

### FIELD

Various aspects of the disclosure relate generally to surgical systems, and more specifically to a surgical system for detecting liquid immersion of an ultrasonic instrument. Other aspects are also described.

### BACKGROUND

Minimally-invasive surgery, MIS, such as laparoscopic surgery, uses techniques that are intended to reduce tissue damage during a surgical procedure. Laparoscopic procedures typically call for creating a number of small incisions in the patient, e.g., in the abdomen, through which several surgical tools such as an endoscope, a blade, a grasper, and a needle, are then inserted into the patient. A gas is injected into the abdomen which insufflates the abdomen thereby providing more space around the tips of the tools, making it easier for the surgeon to see (via the endoscope) and manipulate tissue at the surgical site. MIS can be performed faster and with less surgeon fatigue using a surgical robotic system in which the surgical tools are operatively attached to the distal ends of robotic arms, and a control system actuates the arm and its attached tool. The tip of the tool will mimic the position and orientation movements of a handheld user input device (UID) as the latter is being manipulated by the surgeon. The surgical robotic system may have multiple surgical arms, one or more of which has an attached endoscope and others have attached surgical instruments for performing certain surgical actions.

Control inputs from a user (e.g., surgeon or other operator) are captured via one or more user input devices and then translated into control of the robotic system. For example, in response to user commands, a tool drive having one or more motors may actuate one or more degrees of freedom of a surgical tool when the surgical tool is positioned at the surgical site in the patient.

### SUMMARY

A surgical tool that is used in some MIS procedures is an ultrasonic instrument that uses ultrasonic vibration at its tip to rapidly generate heat for cutting and cauterizing tissue. The tip may include a blade that reaches high temperatures (e.g., greater than 300° C.) during a “heating” cycle in which the blade oscillates against a piece of tissue, thereby producing heat due to friction between the blade and the tissue during the oscillation. After reaching a high temperature, the blade may be used to dissect a portion of tissue, while also sealing the remaining tissue. By performing multiple tasks (e.g., cutting for dissection, cauterizing, etc.), the use of the tool during a laparoscopic surgery reduces instrument exchanges and the number of instruments during the procedure.

The present disclosure provides a laparoscopic surgical system that estimates a temperature of an ultrasonic instrument's blade during the blade's heating and cooling cycles. Specifically, the system may activate the instrument by providing power (e.g., in response to receiving user input by an operator, such as pressing on a petal or button) for the instrument's blade to oscillate, as it is used to dissect tissue. While the instrument is active in this “high-power” state, the system may determine the temperature of the blade based on one or more characteristics (e.g., an input voltage, an input current, a resonance frequency, etc.) of the instrument. After the heating cycle is terminated (e.g., the operator releasing the petal), the system may enter a “low-power” state (or cooling cycle) in which the ultrasonic instrument may draw less power (e.g., to be provided less current) to cause the blade to vibrate less than while the instrument is in the high-power state. While in this low-power state, the instrument may not draw sufficient power to produce frictional heat (e.g., due to the blade vibrating over a lower excursion than needed to produce the heat), but may have sufficient power to determine one or more characteristics of the instrument, such as a resonance frequency of the blade with which the system may use to estimate the temperature of the ultrasonic instrument while it is cooling down. As a result, the system may provide the operator with a (e.g., continuous) temperature reading of the ultrasonic instrument between heating and cooling cycles.

The temperature estimate of the ultrasonic instrument may be affected when the blade of the instrument is contacting tissue and/or immersed in liquid (e.g., blood, saline, etc.). As described herein, the temperature estimate of the blade may be based on the blade's resonance frequency. When the blade is immersed in liquid and/or touching an object, however, the blades resonance frequency may change (e.g., due to the stiffness of the blade increasing). As a result, the surgical system may be unable to effectively estimate the temperature of the end effector. Therefore, there is a need for the surgical system to detect the status of the end effector of the ultrasonic instrument (e.g., whether the end effector is immersed in liquid and/or in contact with an object) in order to properly and effectively estimate the temperature of the end effector.

In addition, there is a need for the surgical system to notify (alert) an operator of the system when at least a portion (e.g., the blade) of the end effector is at least partially submerged in liquid and/or in contact with an object. As described herein, once the ultrasonic instrument switches from the heating cycle to the cooling cycle, the blade may be too hot to touch tissue. During a surgical operation, however, the operator may have minimal visibility of the hot end effector. As a result, the operator may be unable to discern, visually, whether or not the end effector is touching anything as the end effector cools.

The present disclosure provides a surgical system that detects and alerts an operator that the ultrasonic instrument is in contact with an object (e.g., tissue) and/or at least partially submerged (immersed) in liquid. Specifically, the system determines one or more characteristics of the end effector of the ultrasonic instrument (e.g., while is in the low-power state and is cooling down). For example, a characteristic may be an impedance of the end effector. The system determines that the end effector is at least partially submerged within a liquid (and/or in contact with an object, such as tissue) based on the one or more characteristics. In response, the system displays a notification on a display of the surgical system indicating that the end effector is immersed in liquid (and/or in contact with an object). As a result, the system is able to alert an operator of the status of the end effector, in order for the operator to avoid touching things while the blade is still hot.

In one aspect, the one or more characteristics include an impedance of the end effector, where determining that the end effector is at least partially submerged includes determining that the impedance is above an impedance threshold. In another aspect, the one or more characteristics include a resonance frequency of the end effector, where determining that the end effector is at least partially submerged further includes determining that the resonance frequency is below a resonance frequency threshold. In some aspects, the system determines a type of liquid in which the end effector is at least partially submerged based on one or more characteristics, where the notification indicates the type of liquid.

In one aspect, one or more characteristics include an impedance of the end effector, where determining that the end effector is at least partially submerged includes determining that the impedance is above a first threshold, where the system further determines that the end effector is in contact with an object while at least partially submerged inside the liquid in response to the impedance being above a second threshold that is greater than the first threshold. In some aspects, the notification indicates that the end effector is in contact with an object while submerged in liquid.

In another aspect, while the end effector is in air, the system determines a temperature of the end effector and displays the temperature on the display, and, in response to determining that the end effector is at least partially submerged, replaces the temperature with an estimated temperature of the liquid. As a result, the surgical system may estimate and display the temperature of the end effector based on whether the blade is immersed in liquid and/or in contact with an object.

The above summary does not include an exhaustive list of all aspects of the disclosure. It is contemplated that the disclosure includes all systems and methods that can be practiced from all suitable combinations of the various aspects summarized above, as well as those disclosed in the Detailed Description below and particularly pointed out in the claims. Such combinations may have particular advantages not specifically recited in the above summary.

### BRIEF DESCRIPTION OF THE DRAWINGS

The aspects are illustrated by way of example and not by way of limitation in the figures of the accompanying drawings in which like references indicate similar elements. It should be noted that references to “an” or “one” aspect of this disclosure are not necessarily to the same aspect, and they mean at least one. Also, in the interest of conciseness and reducing the total number of figures, a given figure may be used to illustrate the features of more than one aspect, and not all elements in the figure may be required for a given aspect.

FIG. 1 shows a pictorial view of an example surgical system in an operating arena.

FIG. 2 shows a pictorial view of an ultrasonic instrument and a generator according to one aspect of the disclosure.

FIG. 3 shows an end effector of the ultrasonic instrument of FIG. 2.

FIG. 4 is a block diagram of the surgical system according to one aspect.

FIG. 5 is a flowchart of a process for determining whether an end effector of an ultrasonic instrument is at least partially submerged within liquid and/or in contact with an object.

FIG. 6 show several stages of a display of the surgical system that is showing actions performed by the end effector of the ultrasonic instrument and shows a notification based on whether the end effector is submerged in liquid and/or in contact with an object.

FIG. 7 is a flowchart of a process for an aspect of estimating a temperature of the end effector based on detecting that the ultrasonic instrument is at least partially submerged in liquid.

### DETAILED DESCRIPTION

Several aspects of the disclosure with reference to the appended drawings are now explained. Whenever the shapes, relative positions and other aspects of the parts described in a given aspect are not explicitly defined, the scope of the disclosure here is not limited only to the parts shown, which are meant merely for the purpose of illustration. Also, while numerous details are set forth, it is understood that some aspects may be practiced without these details. In other instances, well-known circuits, structures, and techniques have not been shown in detail so as not to obscure the understanding of this description. Furthermore, unless the meaning is clearly to the contrary, all ranges set forth herein are deemed to be inclusive of each range's endpoints.

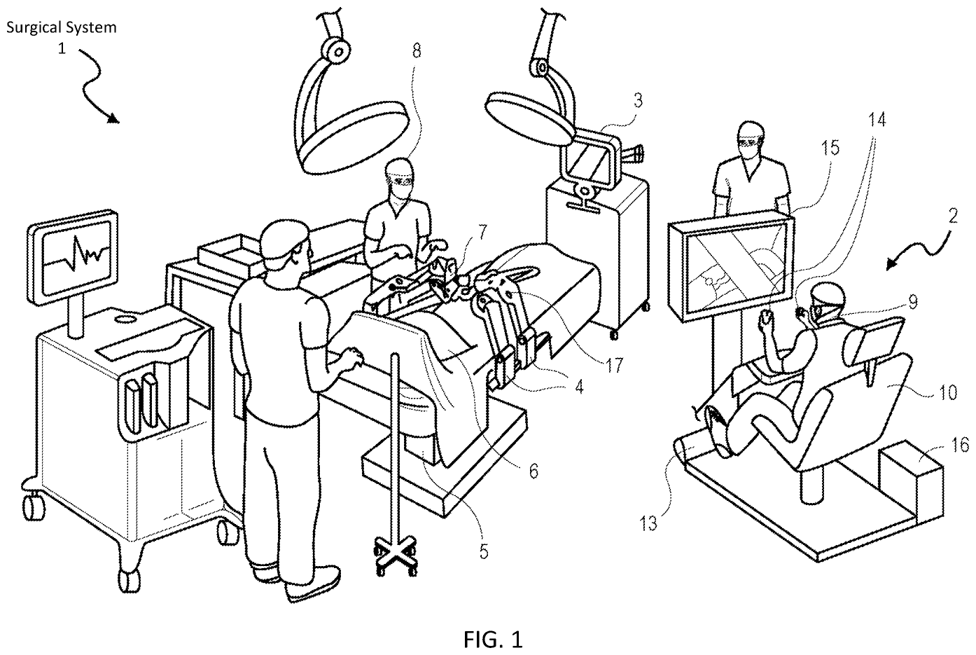

FIG. 1 shows a pictorial view of an example (e.g., laparoscopic) surgical system (which hereafter may be referred to as “system”) 1 in an operating arena. The system 1 includes a user console 2, a control tower 3, and one or more surgical robotic arms 4 at a surgical robotic table (surgical table or surgical platform) 5. In one aspect, the arms 4 may be mounted to a table or bed on which the patient rests as shown in the example of FIG. 1. In one aspect, at least some of the arms 4 may be configured differently. For example, at least some of the arms may be mounted on a ceiling, sidewall, or in another suitable structural support, such as a cart separate from the table. The system 1 can incorporate any number of devices, tools, or accessories used to perform surgery on a patient 6. For example, the system 1 may include one or more surgical tools (instruments) 7 used to perform surgery (surgical procedure). A surgical tool 7 may be an end effector that is attached to a distal end of a surgical arm 4, for executing a surgical procedure.

Each surgical tool 7 may be manipulated manually, robotically, or both, during the surgery. For example, the surgical tool 7 may be a tool used to enter, view, or manipulate an internal anatomy of the patient 6. In an aspect, the surgical tool 7 is a grasper that can grasp tissue of the patient. The surgical tool 7 may be controlled manually, by a bedside operator 8; or it may be controlled robotically, via actuated movement of the surgical robotic arm 4 to which it is attached. For example, when manually controlled an operator may (e.g., physically) hold a portion of the tool (e.g., a handle), and may manually control the tool by moving the handle and/or pressing one or more input controls (e.g., buttons) on the (e.g., handle of the) tool. In another aspect, when controlled robotically, the surgical system may manipulate the surgical tool based user input (e.g., received via the user console 2, as described herein).

Generally, a remote operator 9, such as a surgeon or other operator, may use the user console 2 to remotely manipulate the arms 4 and/or the attached surgical tools 7, e.g., during a teleoperation. The user console 2 may be located in the same operating room as the rest of the system 1, as shown in FIG. 1. In other environments however, the user console 2 may be located in an adjacent or nearby room, or it may be at a remote location, e.g., in a different building, city, or country. The user console 2 may include one or more components, such as a seat 10, one or more foot-operated controls (or foot pedals) 13, one or more (handheld) user-input devices (UIDs) 14, and at least one display 15. The display is configured to display, for example, a view of the surgical site inside the patient 6. The display may be configured to display image data (e.g., still images and/or video). In one aspect, the display may be any type of display, such as a liquid crystal display (LCD), a light-emitting diode (LED) display, an organic LED (OLED) display, etc. In some aspects, the display may be a 3D immersive display that is for displaying 3D (surgical) presentations. For instance, during a surgical procedure one or more endoscopic cameras may be capturing image data of a surgical site, which the display presents to the user in 3D. In one aspect, the 3D display may be an autostereoscopic display that provides 3D perception to the user without the need for special glasses. As another example, the 3D display may be a stereoscopic display that provides 3D perception with the use of glasses (e.g., via active shutter or polarized).

In another aspect, the display 15 may be configured to display at last one graphical user interface (GUI) that may provide informative and/or interactive content, to thereby assist a user in performing a surgical procedure with one or more instruments in the surgical system 1. For example, some of the content displayed may include image data captured by one or more endoscopic cameras, as described herein. In another aspect, the GUI may include selectable UI items, which when manipulated by the user may cause the system to perform one or more operations. For instance, the GUI may include a UI item as interactive content to switch control between robotic arms. In one aspect, to interact with the GUI, the system may include input devices, such as a keyboard, a mouse, etc. In another aspect, the user may interact with the GUI using the UID 14. For instance, the user may manipulate the UID to navigate through the GUI, (e.g., with a cursor), and to make a selection may hover the cursor over a UI item and manipulate the UID (e.g., selecting a control or button). In some aspects, the display may be a touch-sensitive display screen. In this case, the user may perform a selection by navigating and selecting through touching the display. In some aspects, any method may be used to navigate and/or select a UI item.

As shown, the remote operator 9 is sitting in the seat 10 and viewing the user display 15 while manipulating a foot-operated control 13 and a handheld UID 14 in order to remotely control one or more of the arms 4 and the surgical tools 7 (that are mounted on the distal ends of the arms 4.)

In some variations, the bedside operator 8 may also operate the system 1 in an “over the bed” mode, in which the beside operator 8 (user) is now at a side of the patient 6 and is simultaneously manipulating a robotically-driven tool (end effector as attached to the arm 4), e.g., with a handheld UID 14 held in one hand, and a manual laparoscopic tool. For example, the bedside operator's left hand may be manipulating the handheld UID to control a robotic component, while the bedside operator's right hand may be manipulating a manual laparoscopic tool. Thus, in these variations, the bedside operator 8 may perform both robotic-assisted minimally invasive surgery and manual laparoscopic surgery on the patient 6.

During an example procedure (surgery), the patient 6 is prepped and draped in a sterile fashion to achieve anesthesia. Initial access to the surgical site may be performed manually while the arms of the system 1 are in a stowed configuration or withdrawn configuration (to facilitate access to the surgical site.) Once access is completed, initial positioning or preparation of the system 1 including its arms 4 may be performed. Next, the surgery proceeds with the remote operator 9 at the user console 2 utilizing the foot-operated controls 13 and the UIDs 14 to manipulate the various end effectors and perhaps an imaging system, to perform the surgery. Manual assistance may also be provided at the procedure bed or table, by sterile-gowned bedside personnel, e.g., the bedside operator 8 who may perform tasks such as retracting tissues, performing manual repositioning, and tool exchange upon one or more of the robotic arms 4. Non-sterile personnel may also be present to assist the remote operator 9 at the user console 2. When the procedure or surgery is completed, the system 1 and the user console 2 may be configured or set in a state to facilitate post-operative procedures such as cleaning or sterilization and healthcare record entry or printout via the user console 2.

In one aspect, the remote operator 9 holds and moves the UID 14 to provide an input command to drive (move) one or more robotic arm actuators 17 (or driving mechanism) in the system 1 for teleoperation. The UID 14 may be communicatively coupled to the rest of the system 1, e.g., via a console computer system 16 (or host). The UID 14 can generate spatial state signals corresponding to movement of the UID 14, e.g. position and orientation of the handheld housing of the UID, and the spatial state signals may be input signals to control motions of the robotic arm actuators 17. The system 1 may use control signals derived from the spatial state signals, to control proportional motion of the actuators 17. In one aspect, a console processor of the console computer system 16 receives the spatial state signals and generates the corresponding control signals. Based on these control signals, which control how the actuators 17 are energized to drive a segment or link of the arm 4, the movement of a corresponding surgical tool that is attached to the arm may mimic the movement of the UID 14. Similarly, interaction between the remote operator 9 and the UID 14 can generate for example a grip control signal that causes a jaw of a grasper of the surgical tool 7 to close and grip the tissue of patient 6.

The system 1 may include several UIDs 14, where respective control signals are generated for each UID that control the actuators and the surgical tool (end effector) of a respective arm 4. For example, the remote operator 9 may move a first UID 14 to control the motion of an actuator 17 that is in a left robotic arm, where the actuator responds by moving linkages, gears, etc., in that arm 4. Similarly, movement of a second UID 14 by the remote operator 9 controls the motion of another actuator 17, which in turn drives other linkages, gears, etc., of the system 1. The system 1 may include a right arm 4 that is secured to the bed or table to the right side of the patient, and a left arm 4 that is at the left side of the patient. An actuator 17 may include one or more motors that are controlled so that they drive the rotation of a joint of the arm 4, to for example change, relative to the patient, an orientation of an endoscope or a grasper of the surgical tool 7 that is attached to that arm. Motion of several actuators 17 in the same arm 4 can be controlled by the spatial state signals generated from a particular UID 14. The UIDs 14 can also control motion of respective surgical tool graspers. For example, each UID 14 can generate a respective grip signal to control motion of an actuator, e.g., a linear actuator that opens or closes jaws of the grasper at a distal end of surgical tool 7 to grip tissue within patient 6.

In some aspects, the communication between the surgical robotic table 5 and the user console 2 may be through a control tower 3, which may translate user commands that are received from the user console 2 (and more particularly from the console computer system 16) into robotic control commands that transmitted to the arms 4 on the surgical table 5. The control tower 3 may also transmit status and feedback from the surgical table 5 back to the user console 2. The communication connections between the surgical table 5, the user console 2, and the control tower 3 may be via wired (e.g., optical fiber) and/or wireless links, using any suitable one of a variety of wireless data communication protocols, such as BLUETOOTH protocol. Any wired connections may be optionally built into the floor and/or walls or ceiling of the operating room. The system 1 may provide video output to one or more displays, including displays within the operating room as well as remote displays that are accessible via the Internet or other networks. The video output or feed may also be encrypted to ensure privacy and all or portions of the video output may be saved to a server or electronic healthcare record system.

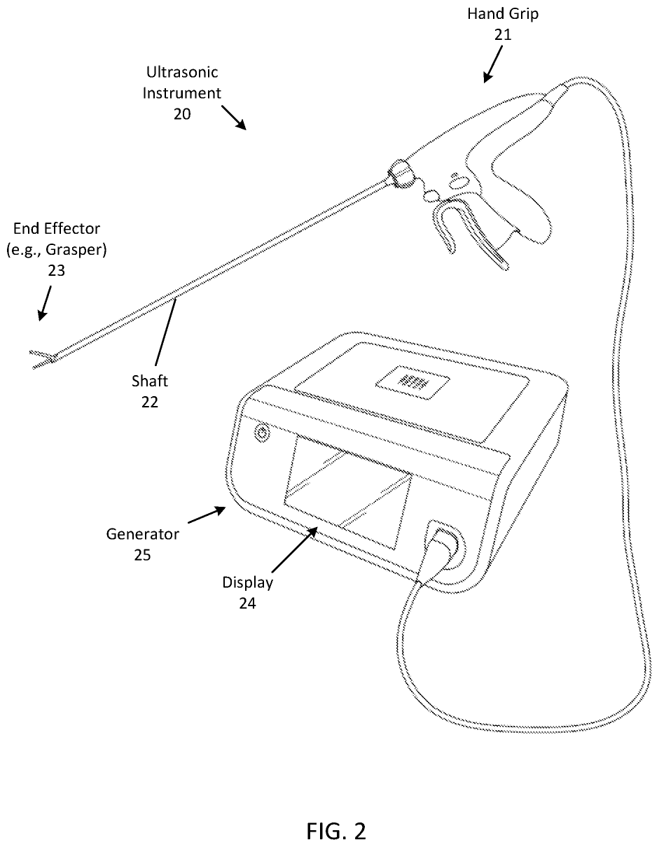

FIG. 2 shows a pictorial view of an ultrasonic instrument 20 and a generator 25 according to one aspect of the disclosure. As shown, the ultrasonic instrument is a hand-held laparoscopic tool that is configured to perform ultrasonic surgical operations (e.g., cutting and sealing tissue) based on manual operations (e.g., of a hand grip 21) of the instrument by an operator (e.g., surgeon). For instance, during a laparoscopic (or endoscopic) surgical procedure, a small incision is made in a patient, and the ultrasonic instrument may be inserted into a cavity of the patient (e.g., a gas is used for insufflation of the cavity), where the end effector may be used by the operator to manipulate tissue and perform surgical operations (e.g., cutting and/or cauterizing, etc.). The ultrasonic instrument is coupled (e.g., via a cable) to the generator (as shown) that enables the ultrasonic instrument to operate in one or more power states, as described herein.

The ultrasonic instrument includes the hand grip (e.g., which includes a tool drive) 21, a shaft (or cannula) 22, and an end effector 23 (e.g., which may be coupled to a shaft of the instrument) that is loaded into the cannula, in accordance with aspects of the subject technology.

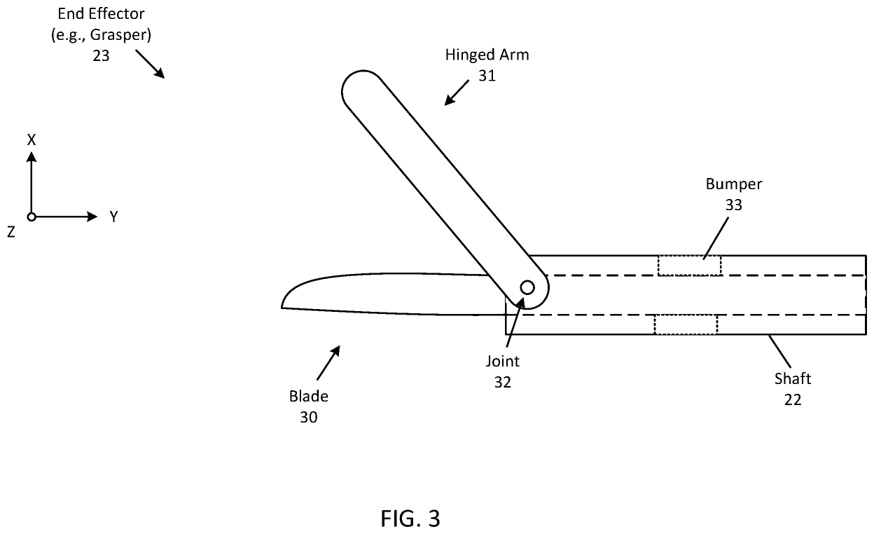

The hand grip 21 is arranged to be held by an operator, and allows the operator to manipulate the (e.g., end effector 23 of the) ultrasonic instrument during a surgical operation. In one embodiment, the hand grip may include one or more inputs (e.g., a trigger, one or more buttons, etc.), that allow an operator to control the ultrasonic instrument. For example, the instrument may include a trigger, which when pulled by one or more fingers of the user while being held produces a control signal that allows the user to control the end effector of the instrument (and/or control a portion of the surgical system). In particular, the trigger may be arranged to manipulate the end effector (e.g., by adjusting the position of the hinged arm 31 shown in FIG. 3). In another aspect, the hand grip may include one or more inputs for changing a power state of the instrument. More about power states of the instrument are described herein.

As described herein, the hand grip may include a tool drive that is arranged to drive the end effector 23 of the ultrasonic instrument. Specifically, the tool drive may include a (e.g., linear) motor or actuator that is arranged to vibrate (or oscillate) the (e.g., blade of the) end effector at one or more frequencies (e.g., at a very high (ultrasonic) frequency, and at a low frequency). In some aspects, the tool drive is configured to vibrate the end effector such that a portion of the end effector (e.g., a blade) moves back and forth along one or more axes. Specifically, the tool drive may vibrate the end effector over one or more excursions, where over each excursion the (e.g., blade of the) end effector may be displaced at a different distance from a starting (or beginning) position. More about how the end effector vibrates is described herein. In another aspect, the tool drive may include an ultrasonic transducer that is configured to vibrate the end effector according to an input voltage/input current (e.g., applied by the generator 25).

As described thus far, the ultrasonic instrument may include the end effector 23 and the hand grip (which includes a tool drive) 21. Specifically, the instrument includes the grip 21, the shaft 22 that is coupled to a distal end of the hand grip, and the end effector 23 that is coupled to a distal end of the shaft. In which case, the ultrasonic instrument as referred herein may be the end effector, which may be coupled to the (e.g., tool drive via the shaft 22 of the) hand grip. In one aspect, the (e.g., end effector of the) ultrasonic instrument may be separate from (and removably coupled to) the hand grip. In some aspects, the shaft receives and guides (e.g., a shaft of) the blade in order to couple to the instrument.

As described herein, the surgical system 1 includes the ultrasonic instrument 20 that is configured to produce heat based on vibrations of its end effector 23. In another embodiment, the instrument may be any type of energy (e.g., endoscopic, laparoscopic, etc.) tool that is designed to generate heat.

As described thus far, the ultrasonic instrument 20 may be a hand-held laparoscopic instrument that may be manually is held and manipulated by an operator. In another embodiment, the instrument may be a part of a surgical robotic arm. Specifically, the ultrasonic instrument may be coupled to a robotic arm and powered by the generator, as described herein. For example, the ultrasonic instrument may be coupled to a distal end of a robotic arm (e.g., arm 4 in FIG. 1), which includes several components that allow the robotic arm to be controlled by an operator. For example, the surgical robotic arm 4 may include a plurality of links and a plurality of actuated joint modules for actuating the plurality of links relative to one another. The joint modules may include various types, such as a pitch joint or a roll joint, which may substantially constrain the movement of the adjacent links around certain axes relative to others. The plurality of the joint modules of the robotic arm 4 can be actuated to position and orient the ultrasonic instrument for robotic surgeries. In one embodiment, the ultrasonic instrument may be coupled to the distal end via a tool drive that is arranged to actuate the end effector 23 of the instrument.

In the case in which the ultrasonic instrument is coupled to a robotic arm, movement and operation of the ultrasonic instrument may be performed via one or more user controls (e.g., UIDs, foot pedals, etc.) that are coupled to the surgical system. For example, a UID may be arranged to open/close the grasper 23 of the ultrasonic instrument, and/or may be arranged to adjust a spatial position (in space) of the grasper based on user input (e.g., the position of the UID).

Turning to FIG. 3, this figure shows the end effector 23 of the ultrasonic instrument of FIG. 2. Specifically, this figure shows that the end effector is a grasper (or grasping device) that includes a blade (or tip) 30 as one jaw and a hinged arm (or jaw) 31 that is rotatably coupled to a joint (or robotic wrist) 32, which is coupled to a distal end of the shaft. In another aspect, the (e.g., joint 32 of the) end effector 23 may be a part of a portion (distal end) of the shaft 22. In one aspect, the grasper (or a part of the grasper) is received through the shaft 22. For instance, the blade may be received (and extend) through the shaft and is arranged to couple to the tool drive (e.g., of the hand grip 21), at (or towards) a proximal end of the shaft. Thus, as shown, the blade extends through the shaft (illustrated as dashed lines), where a portion of the blade (e.g., the tip) extends out of the shaft 22 and into the environment. Inside the shaft are two bumpers 33 that are disposed between the portion of the blade that extends through the shaft and (e.g., an interior side of) the shaft. In one aspect, bumpers may be designed to prevent the blade from coming into contact with the interior side of the shaft (e.g., while the blade is vibrating back and forth). In one aspect, the blade may (at least partially) come into contact (e.g., touch) the bumpers during operation (while the blade oscillates). In another aspect, the interior portion for the blade may not be in contact with (at least one of the) bumpers, as the blade vibrates. In another aspect, the blade may inadvertently come into contact with at least one bumper. For example, during a surgical operation an object may come into contact with the shaft, causing the interior portion of the blade to shift (or move) towards a bumper (due to the impact of the object), causing the blade to come into contact with the bumper. In another aspect, the shaft may not include bumpers. In another aspect, the blade 30 may be coupled to (e.g., a portion of) the shaft 22 (e.g., at a distal end of the shaft). Thus, the internal portion of the shaft, illustrated as dashed lines, may be coupled to the blade 30.

The hinged arm 31 is rotatably coupled (at the joint 32) to the shaft 22, and is arranged to rotate about a rotational (Z-)axis (e.g., in the Z-direction). Specifically, the grasper may be arranged to open and close based on the rotational position of the hinged arm about the rotational axis of the joint with respect to the blade (and/or shaft). For example, the grasper is arranged to open (or is in an opened position) when the hinged arm is rotated away from the blade (e.g., by a threshold distance). While in this position, the end effector may be orientated whereby an object, such as tissue, may be disposed between the blade and the hinged arm (e.g., by moving the end effector about the object). The grasper may be closed (or in a closed position), when the hinged arm rotates towards the blade (e.g., within the threshold distance), whereby the grasper may grab the object between the blade and the hinged arm. As described herein, the hinged arm may be arranged to apply pressure against a grasped object (e.g., squeezing the object between the jaws) in order to grab and/or perform a dissection upon the object. In another aspect, the hinged arm 31 may be rotatably coupled to (a portion of) the blade. In one aspect, the blade 30 and the hinged arm may be received through the shaft such that the arm (and/or the blade) are coupled to another shaft that is passed through the shaft 22.

As described herein, the blade 30 is a jaw of the grasper. In particular, the blade is a jaw that may not rotate (e.g., about the Z-axis) with respect to the end effector. The blade may be arranged to vibrate along a longitudinal (Y-)axis (in the Y-direction) of the blade to produce heat while the ultrasonic instrument is in a high-power state (or mode). In particular, the blade may be driven (e.g., by the tool drive of the hand grip 21) to move back and forth (e.g., linearly) along the longitudinal axis of the end effector (and through the shaft, as described herein), so as to repeatedly displace the blade 30 at a (e.g., constant) frequency. Specifically, the blade may vibrate (e.g., reciprocate back and forth) over an excursion (or displacement) in which the blade moves a distance (e.g., forward or away from the end effector) from a starting position, and then moves the distance back. In one aspect, the excursion may be a distance the blade moves from a starting position to an extended position. In another aspect, the excursion may be the distance the blade moves forward and backward.

As described herein, the blade may produce frictional heat while vibrating against an object. Specifically, the blade may come into contact with tissue while the grasper is squeezing tissue between the two jaws 30 and 31, and may vibrate against the tissue. As the blade vibrates, the end effector may cut and/or cauterize the tissue, as described herein. In one aspect, the blade may vibrate differently (e.g., over different excursions) based on a power state of (e.g., how much power is being provided to) the ultrasonic instrument. More about the vibrating blade and the power states of the ultrasonic instrument are described herein.

As described thus far, the end effector 23 may be a grasper. In another aspect, the end effector may be any type of tool that may be designed to be manipulated by the (e.g., hand grip 21 of the) ultrasonic instrument. For example, the end effector may be an endoscope, a stapler, etc.

Turning back to FIG. 2, the generator 25 is configured to control and provide power to ultrasonic instrument to control (e.g., heat) the end effector 23 while the instrument is coupled to the generator and being used by an operator (e.g., during a laparoscopic surgery to manipulate tissue and/or perform one or more surgical tasks upon tissue, such as to cut and seal vessels and/or to cut, grasp, and dissect tissues). In particular, the generator may provide power to the ultrasonic instrument, such that the (e.g., ultrasonic instrument of the) surgical system 1 may operate in one of one or more power states. For example, the generator may provide power to the instrument such that the ultrasonic instrument is in a “high-power” state (or “heating cycle”) in which the instrument draws power (or current) from the generator (e.g., at a particular voltage) to cause the end effector 23 to produce heat. For example, the generator may provide (e.g., a first) current (or input current) to the (e.g., tool drive of the) hand grip of the ultrasonic instrument, which may use this current to drive the blade 30 to vibrate (or oscillate) over a (first) excursion (and at a particular frequency). Frictional heat may be produced by the end effector while the blade of the end effector is vibrating over this excursion up against an object, such as tissue, as described herein. In another aspect, the ultrasonic instrument may be arranged to operate in a “low-power” state (or “cooling cycle”) in which the ultrasonic instrument no longer draws the (sufficient or as much) power provided by the generator, while the instrument was in the high-power state, to heat the end effector. Specifically, while in this state, the generator may be configured to provide less power to the ultrasonic instrument than the power provided by the generator while instrument was in the high-power state, such that the end effector does not produce heat (e.g., when in contact with an object). In particular, the generator may provide less current (e.g., a second current) to the ultrasonic instrument than the (first) current provided by the generator while the instrument operates in the high-power state, and as a result, this does not cause the end effector to produce heat (or as much heat as when the ultrasonic instrument is in the high-power state). As a result, the ultrasonic instrument may begin to cool, once it enters the low-power state from the high-power state. Ultimately, if kept in the low-power state, the ultrasonic instrument would drop to (at least) a threshold temperature (e.g., room temperature). In one aspect, the second current may be less than a predefined threshold current. In one aspect, the blade may vibrate at a same frequency in the low-power state as in the high-power state. In another aspect, the blade may vibrate the same within a tolerance frequency range.

As a result, of the lesser current provided to the instrument while in the low-power state, the blade of the end effector may be driven differently by the tool drive 21 than when the instrument is in the high-power state. In particular, the blade may vibrate over a different excursion than over which the blade vibrates while the instrument is in the high-power state. For instance, while in the high-power state, the blade may vibrate over the first (e.g., high) excursion, which may cause the blade to produce heat when pressed against an object, whereas, while in the low-power state, the blade may vibrate over a second (lower) excursion, which may be less than the first excursion (e.g., the blade being displaced less along the longitudinal axis than in the first excursion). In some aspects, the second excursion may be less than a minimum threshold (e.g., at which the blade would produce heat if the blade were to vibrate over the minimum threshold). In one aspect, the end effector may not produce frictional heat, while vibrating over this lower excursion and while up against (in contact with) an object (e.g., while the grasper is squeezing the object), such as a blood vessel. In one aspect, the resonant frequency is maintained within a tolerance range regardless of which power state the instrument is operating.

In one aspect, the difference in vibration of the end effector may be based on the amount of power that is being drawn by the ultrasonic instrument while in the different states. For instance, the excursion at which the blade is displaced while it oscillates may be based on (e.g., proportional to) the power drawn by the instrument, whereby more power drawn by the instrument may cause the blade to vibrate over the high excursion. Conversely, while the ultrasonic instrument is in the low-power state the instrument may draw less power that causes the blade to vibrate less (than while the instrument is in the low-power state). As a result of oscillating over a lesser displacement, the blade may not produce frictional heat (e.g., while in contact with tissue). In another aspect, the blade may produce some frictional heat while in the low-power state and in contact with an object, but may be less than the heat produced while the instrument is in the high-power state. In this case, this produced frictional heat may not be enough to cut and/or seal tissue. In some aspects, as a result of operating in the low-power state, the end effector of the ultrasonic instrument may enter a cooling cycle, whereby the heat produced by the end effector while the instrument was in the high-power state dissipates (e.g., over a period of time). In another aspect, the blade may not vibrate (e.g., the tool drive may not drive the blade) while in this low-power state.

In one aspect, the system may enter (or operate in) at least one of the power states based on user input (e.g., received by the generator 25). In particular, the generator may provide power to the ultrasonic instrument based on receiving user input into one or more input devices (e.g., input into a foot petal, an UID that is controlled by an operator and communicatively coupled with the system 1, and/or input at the hand grip 21 of the ultrasonic instrument). The provided power based on the user input may put the ultrasonic instrument in the high-power state in which the ultrasonic instrument draws power from the generator to heat the (e.g., blade 30 of the) end effector 23. For example, when the generator receives (a first) user input (e.g., by the operator pulling on or pressing a trigger on the hand grip 21), the generator may provide current to the (e.g., tool drive of the) ultrasonic instrument, which uses the current to drive the end effector, as described herein. Thus, in the case where the trigger controls the hinged arm of the end effector, the generator is configured to provide the current when the hinged arm is moved (e.g., towards the blade 30 by at least a threshold distance). In another aspect, the system may enter the low-power state based on another (e.g., second) user input (e.g., receiving input from a different input device coupled to the generator, such as a foot pedal).

In some aspects, the ultrasonic instrument may be arranged to switch between the high-power state and the low-power state. As described herein, the instrument may operate in the high-power state while the generator is receiving user input (e.g., the user pulling on or pressing a trigger on the hand grip). The instrument may operate in the low-power state in response to the generator not receiving user input. For instance, the ultrasonic instrument may switch from the high-power state into the low-power state in response to the user releasing the trigger on the hand grip, the generator may transition between the two states). In one aspect, the instrument may operate in the low-power state while the operator is not actively using the instrument to perform ultrasonic instrument operations, as described herein. Specifically, the system may enter the low-power state, while user input is not received into one or more input devices that are used by the operator to enter the high-power state. Once, however, the operator wishes to actively use the ultrasonic instrument, the ultrasonic instrument may switch back into the high-power state (e.g., in response to user input). In another aspect, the instrument may operate in the low-power state in response to receiving user input (e.g., the user pressing a button on a UID). In another aspect, the instrument may operate in this state for a period of time. As described herein, the surgical system is configured to determine a temperature of the end effector while in the low-power state (e.g., after switching from the high-power state) in order to notify an operator of the temperature, which may be high due to the instrumenting having operated in the high-power state. Once the end effector cools to a particular temperature (e.g., equal to or below a predefined temperature), the generator may deactivate the instrument by ceasing to provide the lower current, since at this temperature the end effector may not cause thermal injuries if it were to come into contact with tissue.

In one aspect, the generator may provide different levels of current to heat up the blade, which may be based on user input. For instance, the generator may receive a first user input (e.g., from one petal coupled to the generator) and, in response, provide the ultrasonic instrument with a maximum (allowable) amount of current. The ultrasonic instrument may then drive the end effector over a maximum (e.g., predefined) excursion, which may result in the end effector producing heat at a (first) high temperature. When the generator receives a second user input (e.g., from another petal coupled to the generator), however, the generator may provide a lesser amount of current to the ultrasonic instrument. As a result, the ultrasonic instrument may draw less power to cause the end effector to vibrate over a (second) lower excursion, which may be lower than the first excursion over which the blade vibrates in response to the first user input. This lower excursion, however, may cause the end effector to heat at a lower temperature than the first temperature of the end effector when the ultrasonic instrument draws more current (in response to the generator receiving the first user input). By heating the end effector to different temperatures, different types of tissues may be cut and/or cauterized. For example, fattier tissues may require the end effector to be hotter (having the first temperature), whereas thinner (and less fatty) tissues may require less heat (having the second temperature), in order to cut and/or cauterize the tissues. In another aspect, the generator may be configured to provide one current while in the high-power state (e.g., to drive the end effector over the first high excursion).

As described herein, the ultrasonic instrument may be activated (e.g., operate in the high-power state) based on whether the end effector is in a closed position so as to grasp an object (e.g., a piece of tissue). For example, the ultrasonic instrument may be (e.g., user) activated, such that the ultrasonic instrument may operate in the high-power state so as to draw enough current to cause the end effector to produce heat. In particular, the generator may activate the ultrasonic instrument upon receiving user input to close the end effector (e.g., to cause the hinged arm 31 to move within a distance of the blade 30). Once user input is received to move the hinged arm, the generator may be configured to provide (e.g., enough) power to activate the instrument, as described herein. In some aspects, the generator may activate the instrument based upon a determination that the hinged arm and/or the blade are in contact with an object. For instance, the ultrasonic instrument may include one or more sensors (e.g., force/pressure sensors), that detect a presence of an object and/or detect that an object is in contact with both arms. In particular, upon determining that the grasper is squeezing an object (based on a detected pressure from the sensor being above a threshold), the generator may enter the high-power state. Upon making this determination, the generator may provide the first current to oscillate the blade in order to cause the blade to produce heat. Once the pressure reading drops below the threshold (meaning that the object has been released by the grasper), the generator may switch to the low-power state.

In one aspect, the (e.g., generator of the) surgical system may be configured to determine one or more characteristics of the (end effector of the) ultrasonic instrument, while the instrument is in one or more power states. For example, the generator may be configured to keep track (or monitor) characteristics, such as an input voltage, an input current, a resonance state, a resonance frequency, and/or a (e.g., mechanical) impedance of the (e.g., end effector of the) ultrasonic instrument. In one aspect, the generator may be configured to monitor at least some of these characteristics of the instrument, while the instrument operates in the high-power states. In addition, the system may be configured to determine (at least some of) these characteristics while the instrument is in the low-power state (cooling cycle or cooling period) due to the instrument drawing at least some power. For example, the generator may determine the resonance frequency and the impedance of the (e.g., blade 30 of the) end effector, while in the low-power state. More about determining these characteristics is described herein.

In one aspect, the surgical system may include additional components. For example, the system may include a cable that connects the generator to the ultrasonic instrument (e.g., the ultrasonic transducer, which is configured to convert an electric current drive signal to mechanical vibrations). In one aspect, the ultrasonic transducer may be connected to a waveguide, which is connected to the blade 30 of the end effector 23.

Also shown, the generator 25 also includes a display 24, which is arranged to display information regarding the operation of the ultrasonic instrument. For instance, the display may present temperature information, which state the ultrasonic instrument is currently in, and one or more of the characteristics described herein.

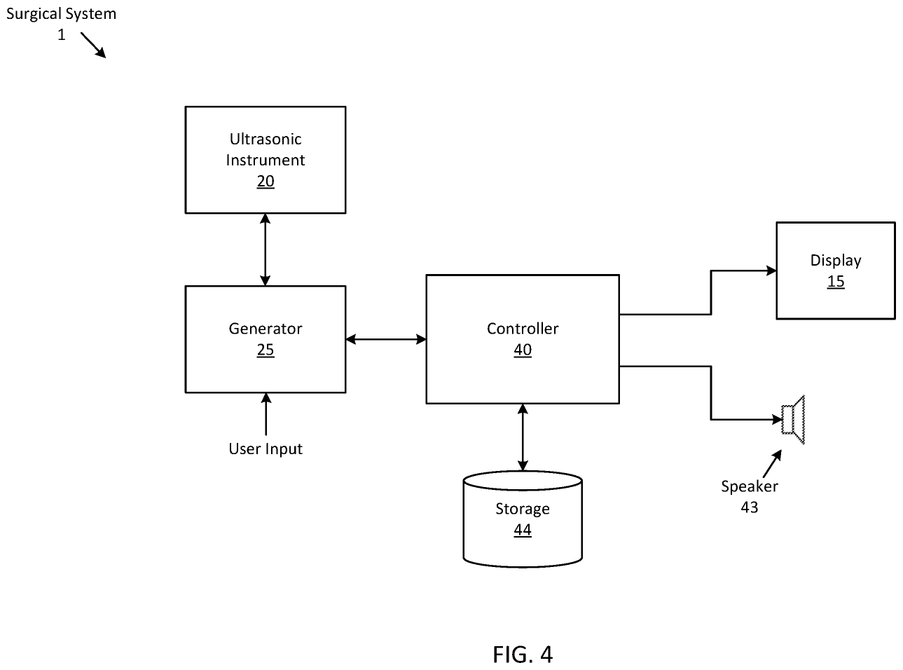

FIG. 4 is a block diagram of the surgical system 1 according to one aspect. The system includes the ultrasonic instrument 20, the generator 25, a controller 40, storage 44, the display 15, and a speaker 43 (that may be a stand-alone speaker or a part of an electronic device of the system, such as the user console 2). In one aspect, the system may include more or less elements, such as having more than one display and/or not having the speaker.

Examples of the storage (e.g., non-transitory machine-readable storage medium) may include read-only memory, random-access memory, CD-ROMS, DVDs, magnetic tape, optical data storage devices, flash memory devices, and phase change memory. Although illustrated as being separate from the controller 40, the storage may be a part of (e.g., internal memory of) the controller.

In some aspects, controller 40 may be a special-purpose processor such as an application-specific integrated circuit (ASIC), a general purpose microprocessor, a field-programmable gate array (FPGA), a digital signal controller, or a set of hardware logic structures (e.g., filters, arithmetic logic units, and dedicated state machines). In one aspect, the controller may be a part an electronic device, such as the console computer system 16, the control tower 3, and/or the user console 2. Although illustrated as being a single component, in one aspect the controller may comprise one or more electronic components (e.g., processors, memory, etc.) that are communicatively coupled on a single electronic device (such as the console computer 16), or across multiple devices (e.g., communicating over a wireless computer network). In some aspects, the controller may be a part of a separate device, such as a part of a remote server that is in communication with one or more electronic devices. In another aspect, the controller may be a part (e.g., at least partially integrated within) the generator 25. In which case, at least some of the other elements (e.g., the speaker and display) may also be a part of (integrated within) the generator. As a result, at least some of the operations performed by the controller described herein may be performed by the generator.

In one aspect, the controller is configured to perform temperature estimation operations for the surgical system 1 to determine a (e.g., real-time) temperature of the (e.g., end effector of the) ultrasonic instrument, while the instrument is in one or more power states (e.g., in the low-power state, where the blade of the end effector is not being actively heated in order to cut and/or seal tissue). Specifically, the controller may determine the temperature based on one or more characteristics of the ultrasonic instrument that are determined while the instrument is in the low-power state, such as a resonance frequency of the (e.g., blade of the) end effector. The controller may determine the temperature using one or more temperature (predefined) models (e.g., which may be stored in storage 44), whereby a temperature model (e.g., polynomial model for the cooling period) may output an estimated temperature of the blade based on (in response to) the resonance frequency, as an input. For example, the controller may determine a normalized change of resonance frequency (e.g., based on a difference between a (predetermined) baseline resonance frequency and a current resonance frequency reading) and determine one or more model coefficients (which are based on the resonance frequency at the beginning of the cooling period and predefined constants). The controller may estimate the temperature by applying the normalized change of resonance frequency and the coefficients to the model as input, which produces the temperature estimate as output.

In one aspect, the controller may be configured to use one or more (predefined) models to determine the temperature of the end effector, while the instrument is in either the heating cycle or the cooling cycle. For example, the controller may be configured to estimate a temperature of the end effector by applying a change in resonance frequency (e.g., a difference between the baseline (or a previously determined) resonance frequency and a current resonance frequency of the end effector) to a hysteresis model (stored in storage 44) that includes a hysteretic relationship between changes in resonance frequency of the end effector and corresponding temperatures of the end effector. In another aspect, the controller may use any method to determine the temperature of the end effector, while the ultrasonic instrument operates in one or more power states.

In one aspect, (at least some) of the temperature estimation operations may be performed by the controller while the end effector is in the cooling period (e.g., while the instrument is in the low-power state). In addition to (or in lieu of) being performed in the cooling period, the temperature estimation operations may be performed while the end effector is “in air”, meaning that the blade is not (at least partially) submerged in liquid and/or is not in contact with an object, such as tissue. In particular, as described herein, during a laparoscopic surgery a cavity may be created within a patient's abdomen using one or more gases. In which case, the temperature estimation operations may be performed while the (blade of the) end effector is within the cavity but not touching tissue and/or liquid. In one aspect, the models used by the controller to estimate the temperature may be predefined in a controlled environment (e.g., a laboratory) while the blade is in the air (e.g., within the patient cavity but not touching an object and not immersed in liquid) during a surgical procedure.

When the blade is either touching an object and/or is at least partially submerged in liquid, the models may be ineffective (or inaccurate) in predicating (estimating) the blade's temperature, due to changes to the blade's characteristics, such as the resonance frequency of the blade. For example, when the blade is touching an object, its resonance frequency (or damped natural frequency, ω_{d}) increases, due to an increase in the blade's stiffness, k. In particular, damped natural frequency may be seen as

ω_{d}=ωₙ\sqrt{1−γ²}

where γ is a damping ratio and ωₙ is the blade's undamped natural frequency, which may be written as dependent on the stiffness of the blade as

$${ω}_{n}^{2} = \frac{k}{m}$$

where m is the mass of the blade. In one aspect, m may include the mass of the blade and any objects (e.g., residual tissue) that is attached to the blade. The damping ratio may be written as

$$γ = \frac{( \frac{b}{m} )}{2 {ω}_{n}}$$

in which b is the damping on the blade. Thus, as k increases, ωₙ increases, and therefore γ decreases, which both lead to an increase in ω_{d}.

Conversely, when the blade is at least partially submerged (immersed) in liquid (e.g., and not touching an object, such as tissue), ω_{d} may decrease due to an increase of the damping ratio, γ, as a result of an increase in the damping, b. If, however, the blade is immersed in liquid and touching an object, ω_{d} may also change due to the increased k and decreased b. Thus, due to the changes in the resonance frequency as the result of touching an object and/or being immersed the controller may be unable to accurately estimate the temperature using model-based methods. As a result, the controller may be configured to determine whether the status of the end effector of the instrument (e.g., whether it is touching an object and/or immersed in liquid), and in response determine (or estimate) a (new) temperature of the end effector based on that determination. More about estimating the temperature is described herein.

In one aspect, at least some of the operations performed by the controller may be implemented in software (e.g., as instructions) stored in memory of the surgical system (e.g., the storage and/or (internal) memory of the controller) and executed by the controller and/or may be implemented by hardware logic structures. In one aspect, at least some of the operations performed by the controller may be performed each time the instrument enters the low-power state (or switches between two or more power states, such as switching between the high-power state to the low-power state).

As shown, the generator may receive user input (e.g., via one or more electronic devices coupled to the generator) for causing the generator to perform one or more operations. For instance, the user input may be received via the ultrasonic instrument (e.g., when the user pulls on a trigger of the hand grip) in order to cause the generator to provide current that causes the ultrasonic instrument to switch from the low-power state to the high-power state, as described herein.

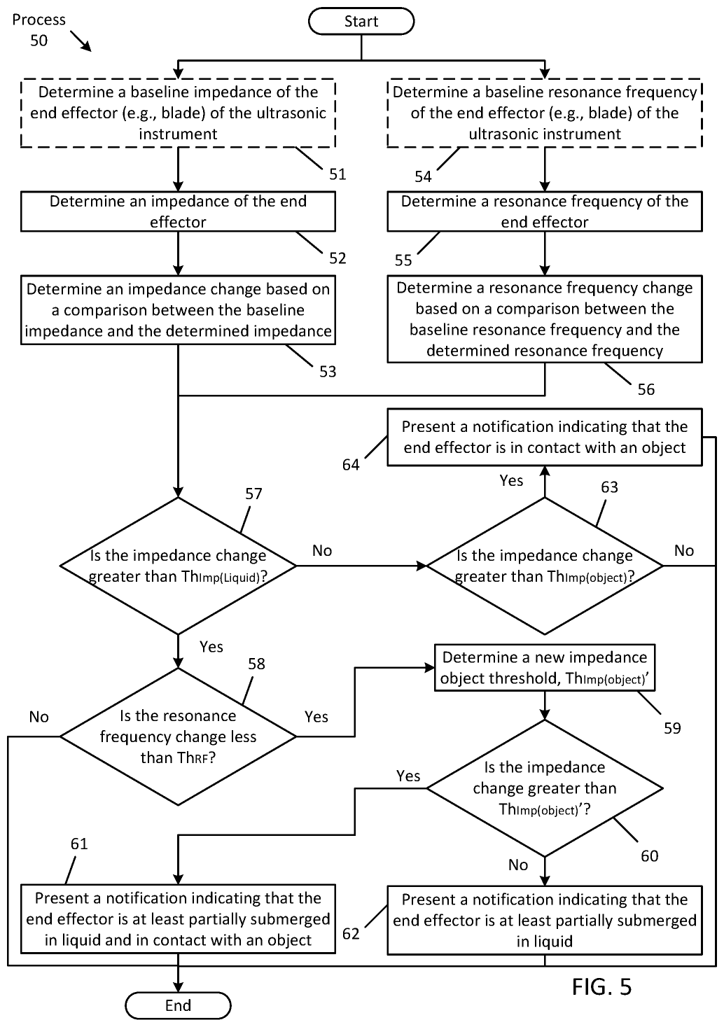

FIG. 5 is a flowchart of a process 50 for determining the status of the end effector, such as whether the end effector 23 of the ultrasonic instrument 20 is at least partially submerged (immersed) within liquid and/or in contact with an object (e.g., tissue). In particular, at least some of these operations may be performed once and/or while the ultrasonic instrument is in one of the one or more power states described herein, such as the low-power state. For example, at least some of the operations may be performed (e.g., each time) the ultrasonic instrument switches from the high-power state to the low-power state, which may be based on user input, as described herein. In which case, the surgical system may perform these operations to determine a status of the end effector, such as whether it is touching an object and/or immersed in liquid. In some aspects, at least some of the operations may be performed periodically while in the low-power state in order to determine the status of the end effector as it is in the cooling cycle. In one aspect, the process may be performed by the surgical system 1. For instance, each of the processes may be performed by the controller 40. As another example, at least some operations may be performed (e.g., by one or more processors of) the generator 25. Thus, this figure will be described with reference to FIG. 4.

The process begins by the controller 40 determining a baseline (or initial) impedance, Imp_{Baseline}, of the end effector (blade) of the ultrasonic instrument (at block 51). As described herein, the impedance may be a mechanical impedance which may be determined by the controller using one or more of the (monitored) characteristics of the ultrasonic instrument. For example, the controller may determine the input current of the ultrasonic instrument (e.g., used to drive the blade of the end effector), and determine the mechanical impedance based on these parameters (e.g., based on Ohm's law). In one aspect, the input voltage may change to maintain the current that may be set to compensate for changes in the impedance. In one aspect, the controller may determine the impedance by applying one or more of the characteristics into a (predefined) impedance model (e.g., an electro-mechanical model of the impedance of blade), which outputs the mechanical impedance. In another aspect, the controller may use any known method to determine the impedance of the blade. In one aspect, the baseline impedance may be determined at an initial time, to, such as when the ultrasonic instrument 20 is coupled (e.g., plugged into) the generator 25. For instance, once the instrument is plugged into the generator, the controller may perform one or more diagnostic operations upon the instrument (e.g., to determine one or more characteristics, as described herein) to determine Imp_{Baseline}. In another aspect, the generator may be configured to determine Imp_{Baseline}. Thus, based on the operations, the generator may determine the baseline impedance of the end effector's blade, and provide the impedance to the controller.

In some aspects, this baseline impedance may be determined while the end effector is at (or approximately) room temperature (e.g., a temperature between 20-25° C.) and/or while the end effector is in air (e.g., while the blade of the end effector is not touching an object). In another aspect, the baseline impedance may be determined once and stored in the storage 44 (or memory of the controller 40 of) the surgical system 1. For instance, the baseline impedance may be determined a first time the instrument is coupled to the generator. In another aspect, the baseline impedance may be determined every time the ultrasonic instrument is plugged into the generator. In another aspect, the baseline impedance may be determined at start up (e.g., during initial powering up) of the (e.g., ultrasonic instrument by the) surgical system. In another aspect, the baseline impedance may be an impedance that was previously determined (e.g., during a previous performance of the process 50).

In one aspect, the baseline impedance, Imp_{Baseline}, may be determined to be an impedance level of the end effector when the ultrasonic instrument is in the low-power state, the end effector is in an open position, and/or the (e.g., end effector and shaft of the) ultrasonic instrument is not in contact with any objects. In another aspect, the baseline impedance may be determined when (e.g., every time) the ultrasonic instrument enters a cooling period. In some aspects, Imp_{Baseline} may vary between different devices. Moreover, Imp_{Baseline} may drift or step down/up after repeated activations (repeated switching between heating and cooling cycles) throughout a surgical procedure. Since the change in the impedance, ΔImp (from the baseline impedance level) is what the controller uses for detecting whether the end effector is in contact with an object, the controller may be configured to update the baseline impedance value periodically to prevent erroneous object contact detection due to impedance drift or changes due to repeated activations.

The controller 40 determines an impedance, Imp, of the end effector (at block 52). For instance, the impedance may be a “current” impedance, which is being determined by the controller during a surgical operation (while the instrument is being used by an operator and is in the cooling cycle). In one aspect, the controller may determine Imp using similar (or the same) operations as used to determine the baseline impedance. In some aspects, the impedance determined at this point may be determined after the baseline impedance. The controller determines an impedance change, ΔImp, based on a comparison between the baseline impedance and the determined impedance (at block 53). In particular, the impedance change may be the difference between the two impedances, where ΔImp=Imp−Imp_{Baseline}.

The controller 40 determines a baseline resonance frequency of the end effector (e.g., blade) of the ultrasonic instrument (at block 54). For instance, the controller may determine the baseline resonance, RF_{Baseline}, of the blade at to, as described with respect to the baseline impedance. In one aspect, the controller may determine RF_{Baseline} (at least partially) contemporaneously as (or simultaneously when) Imp_{Baseline} is determined. Thus, RF_{Baseline} may be determined in similar conditions as Imp_{Baseline} (e.g., being determined when the instrument is plugged into the generator, determine at room temperature, etc.).

In some aspects, the generator determines the resonance frequency electronically. For example, the generator may sense voltage and current waveforms (and the difference in phase angle between the two waveforms) that are used to drive the blade of the end effector. Specifically, the ultrasonic instrument 20 (e.g., the tool drive) may include an ultrasonic transducer that is configured to vibrate the blade according to the input voltage and current waveforms. The frequency that produces a difference in phase angle of a threshold (e.g., zero) is the resonance frequency. In one aspect, the generator continues to drive the ultrasonic transducer in resonance and may adjust the output voltage (which may be called phase lock) to continue to drive in resonance (as resonance frequency changes with changes in temperature). In another aspect, the controller 40 may adjust the output frequency. In another aspect, other known methods may be used to determine the resonance frequency.

In one aspect, (at least some) the operations performed in blocks 51 and/or 54 blocks may be omitted from the process 50. For example, as described herein, at least some of these operations may be performed each time the ultrasonic instrument enters the low-power state. The determination of the baseline impedance and/or baseline frequency, however, may be performed one time (e.g., during the initial powering up), in some aspects. As a result, the process 50 may omit either (or both of these) operations in subsequent (at least partial) performances of this process. Thus, blocks 51 and 54 may be optional (e.g., as illustrated as being boxes with dashed boundaries).

The controller 40 is configured to determine a (current) resonance frequency, RF, of the end effector (at block 55). Specifically, the controller may determine RF similarly as the baseline resonance frequency, but RF may be determined after the baseline, similarly as Imp with respect to Imp_{Baseline}. In one aspect, the controller may be configured to determine the impedance (at block 52) and/or the resonance frequency (at block 55) based on a configuration of the ultrasonic instrument. For instance, these characteristics may be determined by the surgical system once (or in response to) the end effector of the instrument is in the open position (e.g., and while also operating in the low-power state). In another aspect, the characteristics may be determined after (or immediately or within a period of time when) the controller determines that the end effector is in the open position. In another aspect, the controller may determine either of these characteristics once the ultrasonic instrument switches between state and/or may determine the characteristics periodically after entering a state. The controller determines a resonance frequency change, ΔRF, based on a comparison between the baseline resonance frequency and the determined resonance frequency (at block 56). Specifically, the controller may determine the change based on a difference between the frequencies, such that ΔRF=RF−RF_{Baseline}. In one aspect, the difference may represent the resonance frequency drift from (or change between) the baseline (or nominal) resonance frequency to the determined resonance frequency of the blade.

The controller 40 determines whether the impedance change, ΔImp, is greater than (and/or equal to) an impedance liquid threshold, Th_{Imp(Liquid)}, (at decision block 57). In one aspect, at least partial immersion of the (end effector of the) ultrasonic instrument may result in an increase in the mechanical impedance, Imp (and therefore an increase in ΔImp). In particular, when the ultrasonic instrument is in the low-power state in which current into the instrument is controlled (e.g., set to a predefined value), the voltage may change due to the immersion in order to maintain the specified current. As a result, if the end effector were at least partially immersed within liquid when the mechanical impedance was determined (e.g., at block 52), the change in voltage may result in an increase in the determined impedance. In one aspect, Th_{Imp(Liquid)} may be a predefined threshold (e.g., a threshold determined in a controlled environment).

If ΔImp<Th_{Imp(liquid)}, meaning that the (e.g., blade 30 of the) end effector is not at least partially submerged (e.g., no part of the blade is submerged within liquid), the controller 40 determines if the impedance change is greater than an impedance object threshold, Th_{Imp(Object)} (at block 63). In particular, the controller is determining whether at least a portion of the end effector is in contact with (touching) an object, such as tissue, a vein, etc., during a surgical procedure based on whether ΔImp<Th_{Imp(Object)}. In one aspect, Th_{Imp(Object)} may be a threshold that is greater than Th_{Imp(liquid)}. In another aspect, Th_{Imp(Object)} may be a threshold that is less than Th_{Imp(liquid)}. If so, the controller presents a notification indicating that the end effector is in contact with an object (at block 64). For example, the system 1 may display a pop-up notification on display 15 indicating that the (e.g., ultrasonic instrument that is being used by the) operator is in contact with an object. As another example, the controller 40 may output an audible notification through the one or more speakers (e.g., speaker 43). For instance, the audible notification may be one or more sounds (e.g., a beep), which indicates that the end effector is in contact with an object. In another aspect, the notification may be a spoken word (e.g., “The Blade is in Contact with an Object!”). In another aspect, any type of notification may be presented.

Returning to decision block 57, if ΔImp>Th_{Imp(liquid)}, the controller determines whether the resonance frequency change, ΔRF, is less than (and/or equal to) a resonance frequency threshold, TH_{RF} (at decision block 58). Specifically, the controller may determine whether ΔRF<Th_{RF}. During an end effector (at least partial) immersion in liquid, the impedance of the instrument may increase, whereas the resonance frequency of the end effector may decrease. In one aspect, the controller may determine that the end effector is immersed within (or in contact with) liquid based on an increase in impedance (e.g., ΔImp>Th_{Imp(liquid)}) and the decrease in resonance frequency (e.g., ΔRF<Th_{RF}) over a (same) period of time (e.g., a period of time in which both characteristics are monitored by the generator). Thus, if these changes occur (e.g., simultaneously or contemporaneously, such as within a period of time) it may be determined that the end effector is in contact with liquid or has been immersed in liquid.

If ΔRF<Th_{RF}, the controller determines a new impedance object threshold, Th_{Imp(Object)}′ (at block 59). In one aspect, the new object threshold may be greater than the object threshold of decision block 63, such that Th_{Imp(Object)}′>Th_{Imp(Object)}. In one aspect, the controller is determining a new threshold to be used in determining whether the end effector that is at least partially submerged within liquid is also in contact with an object (e.g., during a surgical procedure the end effector may be grasping tissue that is immersed in saline). The controller may make this determination based on changes in the impedance. In one aspect, the controller may increase the impedance object threshold based on the threshold liquid impedance. For example, the controller may increase the threshold by (at least) the liquid threshold, such that Th_{Imp(Object)}′=Th_{Imp(Object)}+Th_{Imp(Liquid)}. In another aspect, the new threshold impedance may be determined by increasing Th_{Imp(Object)} by a predetermined amount.

The controller 40 determines whether the impedance change is greater than the new impedance object threshold, Th_{Imp(Object)}′ (at decision block 60). In one aspect, the impedance change is ΔImp, which was determined at block 53. In another aspect, the controller may determine a new impedance change, ΔImp′, and use this to make the determination. If the impedance change is less than the new impedance object threshold, the controller 40 presents a notification indicating that the end effector is at least partially submerged in liquid (at block 62). For example, the controller may display text and/or an image on the display 15 that indicates that the end effector is immersed in liquid, such as an image of a raindrop. If, however, the impedance change is greater than the new impedance object threshold, the controller 40 presents a notification indicating that the end effector is at least partially submerged in liquid and (at least partially) in contact with an object (at block 61). For example, the notification may be a pop-up notification with the image of a raindrop and text indicating that the end effector is in contact with an object (e.g., displaying text reading “Tissue Contact”).

Some aspects may perform one or more variations to the process 50 described herein. For example, the specific operations of the process may not be performed in the exact order shown and described. The specific operations may not be performed in one continuous series of operations and different specific operations may be performed in different aspects. As described herein, the controller may perform at least some of the operations of this process to determine whether the end effector is immersed in liquid and/or in contact with an object. In some aspects, the controller may determine that the end effector is not immersed in liquid and/or not in contact with an object based on the performed operations. For example, in response to the resonance frequency change being greater than Th_{RF}, the controller may determine that the end effector is not (at least partially) immersed in liquid.

In one aspect, at least some of the operations described herein may be performed contemporaneously. For instance, the controller may determine whether the impedance change is greater than Th_{Imp(Liquid)} (at block 57) and determine whether the resonance frequency change is less than Th_{RF}, at (at least partially) the same time. In which case, the controller may determine that the end effector is not immersed in liquid in response to at least one of the characteristics not satisfying the conditions described herein.

In one aspect, the controller 40 may be configured to determine a type of liquid in which the end effector is at least partially submerged based on one or more of the characteristics (e.g., impedance and/or resonance frequency) of the ultrasonic instrument. For example, blood may be more viscous than saline, which may result in Imp being greater when the end effector is immersed in blood than Imp if the end effector was immersed in saline. Thus, the controller may determine the type of liquid by comparing Imp (and/or ΔImp) to a threshold. For example, the controller may perform a table lookup using Imp into a (predefined) data structure that associates impedance (ranges) with types of liquid. The controller may determine the type of liquid that has an associate impedance that is (similar or the same as) Imp. In one aspect, the presented notification may indicate the type of liquid (e.g., indicating that the end effector is immersed in blood).

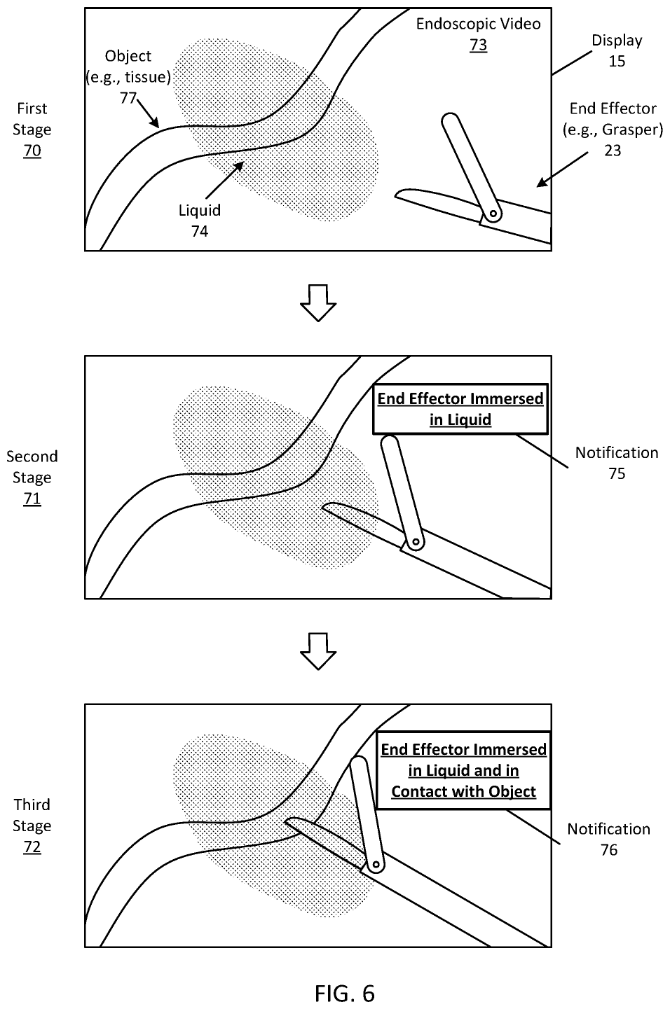

FIG. 6 show several stages of a display of the surgical system that is showing actions performed by the end effector of the ultrasonic instrument and shows a notification based on whether the end effector is submerged in liquid and/or in contact with an object. Specifically, each of the three stages 70-72 is showing a display 15 (and/or display 24 of the generator 25), which is displaying an endoscopic video 73 that is showing an end effector 23 and a portion of an object 77 (e.g., a tissue, such as a blood vessel) that is submerged in liquid 74. In one aspect, the display showing the endoscopic video may be a different display of the surgical system, such as display 24 of the generator 25. In some aspects, the display may show other content, such as other video content and/or a graphical user interface (GUI) of the surgical system that is displaying one or more UI items (e.g., associated with a surgical procedure that is being performed by an operator of the system).

The first stage 70 shows the end effector 23 in front of the liquid 74 and the tissue 77. In one aspect, the end effector may be in the air (e.g., within a cavity of a patient during a surgical procedure). In one aspect, the end effector may be in the low-power state in which the end effector is hot, but is in a cooling cycle (e.g., cooling down). In one aspect, at this stage the controller may determine the baseline impedance and/or baseline resonance frequency.

The second stage 71 shows that (e.g., a portion of the blade 30 of) the end effector 23 has entered (moved forward) and is partially submerged within the liquid 74. In this case, the controller may determine that the end effector is in liquid based on changes to one or more characteristics of the ultrasonic instrument. For instance, once the blade 30 of the end effector has entered the liquid, a measured impedance of the blade may increase and a measured resonance frequency of the blade may decrease, as described herein. As a result, the controller may determine that the blade is immersed in the liquid 74. In addition, this stage shows that, in response to the controller determining that the end effector is in the liquid, a notification 75 is displayed on the display 15 (e.g., overlaid on top of the endoscopic video 73) that reads “End Effector Immersed in Liquid” in order to alert the operator of the status of the end effector, which may be out of view of the operator.

The third stage 72 shows that the blade 30 of the end effector 23 has moved further forward (from as shown in the second stage 71), and is now touching the tissue 77, while also being immersed in the liquid 74. In particular, the controller has made this determination based on additional changes to the characteristics, such as the impedance of the end effector. This stage also shows that the display 15 is showing a notification 76 that indicates that the end effector is immersed in liquid and is touching an object by including the text “End Effector Immersed in Liquid and in Contact with Object” in order to alert the operator of the status of the instrument.

Thus, this figure is illustrating how the controller may continuously perform at least some of the operations of process 50 shown in FIG. 5 to (continuously) monitor and update the operator of the status of the instrument.

As described herein, the surgical system may be configured to determine (estimate) and present (e.g., display on the display 15) a temperature of the (e.g., blade of the) end effector based on one or more of the monitored characteristics of the ultrasonic instrument, such as the resonance frequency, while the instrument is in one of the power states (e.g., the low-power state). Specifically, the (controller 40 of the) system 1 may estimate the temperature according to at least one temperature model, which is configured to determine the temperature of the end effector that is in the air. For example, the temperature model may estimate the temperature based on the resonance frequency of the oscillating blade of the end effector, while the instrument is in the low-power state. If, however, the blade were to touch an object and/or be immersed in liquid, the resonance frequency may be adversely effected (e.g., the resonance frequency may increase while the blade is touching an object due to an increase in the blade's stiffness, k, as described herein. As a result, the temperature model may be unable to effectively estimate the temperature of the blade. Therefore, the system may be configured to estimate the temperature of the end effector based on the status of the instrument (e.g., whether it is touching an object and/or submerged in liquid).

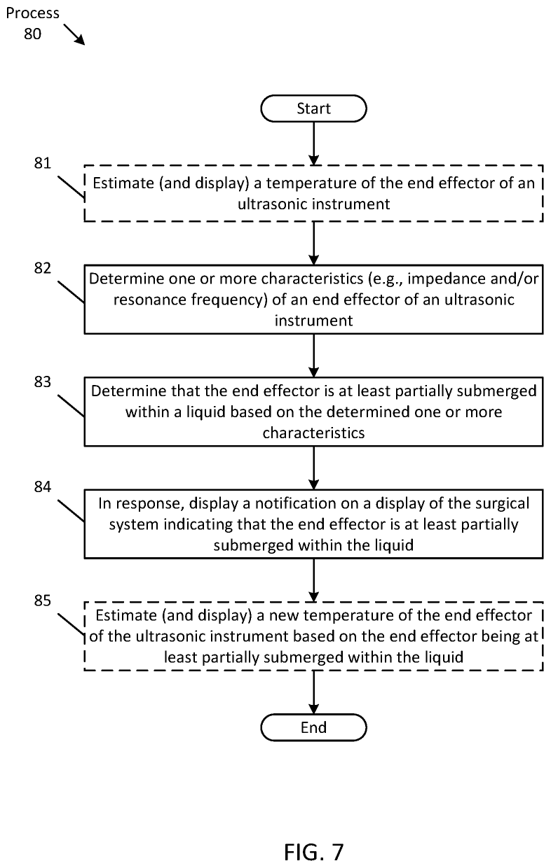

FIG. 7 is a flowchart of a process 80 for an aspect of estimating a temperature of the end effector based on detecting that the ultrasonic instrument is at least partially submerged in liquid. Specifically, the operations may be performed by the controller 40 (and/or the generator 25) of the surgical system 1. The process 80 begins by the controller 40 estimating (and displaying on a display, such as display 15 of FIG. 4) a temperature of the end effector of the ultrasonic instrument (at block 81). Specifically, the controller may determine one or more characteristics of the instrument, such as a resonance frequency of the end effector, and then apply the frequency to a predefined temperature model, which outputs a temperature estimate of the end effector. In one aspect, the estimation of the temperature may be performed while the instrument is in the air (e.g., not touching an object and/or immersed in liquid). In some aspects, the temperature estimate may be determined while the instrument is in the cooling cycle (e.g., in the low-power state).

The controller 40 determines one or more characteristics of the end of the ultrasonic instrument (at block 82). Specifically, the controller may determine the impedance and/or the resonance frequency (e.g., from the generator), as described herein. The controller determines that the end effector is at least partially submerged within a liquid based on the determined one or more characteristics (e.g., at block 83). For instance, the controller may determine that the blade of the end effector is immersed in liquid in response to determining that a change in impedance, ΔImp, is greater than an impedance liquid threshold, Th_{Imp(Liquid)}, and/or in response to determining that a change in resonance frequency, ΔRF, is less than a threshold, Th_{RF}, as described in FIG. 5. In response to determining that the end effector is at least partially submerged, the controller displays a notification on a display of the surgical system indicating that the end effector is at least partially submerged within the liquid (at block 84). For instance, the system may display a notification, such as notification 75 in FIG. 6.

The controller estimates (and displays) a new temperature of the end effector of the ultrasonic instrument based on the end effector being at least partially submerged within the liquid (at block 85). Specifically, the controller may replace the estimated temperature that is displayed on a display of the system (as described with block 81) with a new temperature estimate. In one aspect, the new temperature estimate may be a temperature associated with the liquid in which the end effector is submerged. In particular, with the blade being submerged within the liquid any residual heat of the blade would quickly be dissipated within the liquid. This is due to most liquids having a high thermal capacity, as compared to air. As a result, the blade (within a period of time) will cool down and reach a thermal equilibrium with the liquid in which the blade of the temperature would (approximately) be the same temperature as the liquid. Thus, in response to determining that the end effector is at least partially submerged in liquid, the controller may estimate the temperature of the end effector as being the temperature of the liquid.

In one aspect, the controller may estimate the new temperature based on a determination of the liquid. For example, as described herein, the controller may be configured to determine the type of liquid in which the end effector is submerged (e.g., based on changes in the resonance frequency of the end effector). In which case, the controller may be configured to determine the new temperature based on a determination of the type of liquid in which the end effector is submerged. For instance, the controller may perform a table lookup into a data structure that associates types of liquids with temperature. In another aspect, the controller may determine the temperature of the liquid through other methods. For example, the surgical system may include a temperature sensor that is configured to detect the temperature of the liquid. In another aspect, the temperature of the liquid may be a predefined liquid (e.g., across one or more types of liquids).

Some aspects may perform variations to the process 80 described herein. For example, the specific operations of at least some of the processes may not be performed in the exact order shown and described. The specific operations may not be performed in one continuous series of operations and different specific operations may be performed in different aspects. For example, the operations within dashed boxes may be optional operations that may not be performed while a respective process is performed. In another aspect, one or more operations (which may or may not have dashed boxes may be optional. In one aspect, at least some of the operations described herein (e.g., performed in one or more processes described herein) may be performed automatically (e.g., without user interference). As described herein, the operations of process 80 are used to determine whether the end effector is at least partially submerged and, in response, estimate a new temperature. In some aspects, at least some of these operations may be performed based on whether the end effector is in contact with an object. For example, in lieu of (or in addition to) determining whether the end effector is immersed, the controller may determine whether the end effector is in contact with an object based on one or more characteristics (e.g., impedance) of the end effector (e.g., at block 83). In response to determining that the end effector is touching an object, the controller may be configured to display a notification and estimate (and display) a new temperature (e.g., in the notification) of the end effector based on it touching an object. In this case, the controller may be configured to estimate the temperature of the end effector as being (e.g., approximately) a temperature of the object that the end effector is touching.

In another aspect, the controller may estimate the temperature of the end effector that is touching the object based on an amount of time it is in contact with the object. For instance, as the end effector is touching the object, the temperature may change at a predefined (e.g., linear) rate. In which case, the controller may estimate the temperature based on the predefined rate and the time the end effector touches the object. In one aspect, the controller may continuously update the displayed new temperature based on how long the end effector touches the object. In another aspect, the controller may determine the temperature based on a temperature (linear) model that adjusts the temperature linearly based on the estimate of the temperature (prior to touching the object). In some aspects, the controller may estimate the new temperature of the end effector as being the temperature of the liquid upon a determination that the end effector is immersed in liquid and is touching an object. In some aspects, once the blade is immersed in liquid, the temperature of the blade may be estimated to the temperature of the liquid regardless of whether or not the blade is in contact with an object.

As described herein, the controller may estimate and display the temperature of the end effector. In one aspect, the temperature of the end effector (or an indication of the temperature) may be displayed as a separate notification than (or in the same) notification that indicates that the end effector is immersed in liquid (and/or is in contact with an object). In another aspect, the notification of the status of the end effector may be based on the temperature. For example, upon determining that the end effector is in contact with an object, the controller may be configured to determine whether the temperature estimate of the end effector (e.g., while in air and before in contact with the object) is above a threshold. If so, the displayed notification may alert the operate that the end effector is too hot to touch the object.

In some aspects, the controller may be configured to estimate the temperature of the end effector using one or more of the storage models described herein, in response to determining that the end effector is no longer (at least partially) submerged within liquid (and/or in contact with an object). For instance, the controller may be configured to monitor the one or more characteristics of the end effector to determine whether the end effector is no longer submerged. For example, upon determining that the change in impedance is less than the impedance liquid threshold and/or upon determining that the change in resonance frequency is greater than the resonance frequency threshold, the controller may determine that the end effector is no longer submerged (e.g., and is in air). In response, the controller may be configured to estimate and display the temperature of the end effector, using one or more models describe herein, and/or may remove the displayed notification indicating that the end effector is immersed.

In some aspects, the controller may update Imp_{Baseline} (e.g., in real-time) by continuously (or within a period of time) monitoring the impedance level, Imp (e.g., determining the level of impedance one or more times over the time period) in order to prevent erroneous triggering of object contact due to shifts. Specifically, to update the baseline impedance, the controller may be configured to determine one or more impedances of the end effector over a period of time, and may be configured to determine whether a change in impedance across the one or more impedances throughout the period of time is less than a threshold. In particular, the controller may determine whether one or more impedances remain within a steady state for the time period, Δt. If the change in impedance throughout Δt is below a threshold, ε, the controller may define Imp_{Baseline} based on the change in impedance. In one aspect, in response to determining that the change is less than the threshold, the controller may use the one or more impedances to determine the Imp_{Baseline}. For example, the controller may define Imp_{Baseline} as an average impedance across the one or more impedances, such that if

|Imp(t)−Imp(t+Δt)|<∈

then,

Imp_{Baseline}=mean(Imp(t),Imp(t+1), . . . ,Imp(t+Δt))

In some aspects, the controller may update the baseline impedance when the ultrasonic instrument switches from a heating cycle into a cooling cycle. In particular, the controller may determine that the ultrasonic instrument is in a heating cycle (e.g., the end effector being in a closed position), and in response to determining that the ultrasonic instrument has returned to the cooling cycle (e.g., the end effector now being in the open position and/or in air), the controller may (e.g., begin) to monitor impedance of the end effector over the period of time to determine a new baseline impedance. In some aspects, the new baseline impedance may be different than a previously determined baseline impedance. This may be due to differences in monitored impedance, which may be due to various factors (e.g., how long the end effector was in the heating cycle before returning to the cooling cycle, etc.).

As previously explained, an aspect of the disclosure may be a non-transitory machine-readable medium (such as microelectronic memory) having stored thereon instructions, which program one or more data processing components (generically referred to here as a “processor”) to (automatically) perform ultrasonic instrument operations, temperature estimation operations, and/or liquid immersion and/or object contact detection operations, as described herein. In other aspects, some of these operations might be performed by specific hardware components that contain hardwired logic. Those operations might alternatively be performed by any combination of programmed data processing components and fixed hardwired circuit components.

While certain aspects have been described and shown in the accompanying drawings, it is to be understood that such aspects are merely illustrative of and not restrictive on the broad disclosure, and that the disclosure is not limited to the specific constructions and arrangements shown and described, since various other modifications may occur to those of ordinary skill in the art. The description is thus to be regarded as illustrative instead of limiting.

In some aspects, this disclosure may include the language, for example, “at least one of [element A] and [element B].” This language may refer to one or more of the elements. For example, “at least one of A and B” may refer to “A,” “B,” or “A and B.” Specifically, “at least one of A and B” may refer to “at least one of A and at least one of B,” or “at least of either A or B.” In some aspects, this disclosure may include the language, for example, “[element A], [element B], and/or [element C].” This language may refer to either of the elements or any combination thereof. For instance, “A, B, and/or C” may refer to “A,” “B,” “C,” “A and B,” “A and C,” “B and C,” or “A, B, and C.”

What is claimed is:

1. A method performed by a surgical system, the method comprising:
determining one or more characteristics of an end effector of an ultrasonic instrument;
determining that the end effector is at least partially submerged within a liquid based on the determined one or more characteristics; and
in response, displaying a notification on a display of the surgical system indicating that the end effector is at least partially submerged within the liquid.

2. The method of claim 1, wherein the one or more characteristics comprises an impedance of the end effector, wherein determining that the end effector is at least partially submerged comprises determining that the impedance is above an impedance threshold.

3. The method of claim 2, wherein the one or more characteristics comprises a resonance frequency of the end effector, wherein determining that the end effector is at least partially submerged further comprises determining that the resonance frequency is below a resonance frequency threshold.

4. The method of claim 1 further comprising determining a type of liquid in which the end effector is at least partially submerged based on the one or more characteristics, wherein the notification indicates the type of liquid.

5. The method of claim 1, wherein the one or more characteristics comprises an impedance of the end effector, wherein determining that the end effector is at least partially submerged comprises determining that the impedance is above a first threshold, wherein the method further comprises determining that the end effector is in contact with an object while at least partially submerged inside the liquid in response to the impedance being above a second threshold that is greater than the first threshold.

6. The method of claim 5, wherein the notification indicates that the end effector is in contact with an object while submerged in liquid.

7. The method of claim 1 further comprises
while the end effector is in air,
determining a temperature of the end effector, and
displaying the temperature on the display; and
in response to determining that the end effector is at least partially submerged, replacing the temperature with an estimated temperature of the liquid.

8. A surgical system comprising:
an ultrasonic instrument with an end effector;
a display;
a processor; and
memory having instructions which when executed by the processor causes the surgical system to
determine one or more characteristics of the end effector of the ultrasonic instrument,
determine that the end effector is at least partially submerged within a liquid based on the determined one or more characteristics, and
in response, display a notification on the display indicating that the end effector is at least partially submerged within the liquid.

9. The surgical system of claim 8, wherein the one or more characteristics comprises an impedance of the end effector, wherein the instructions to determine that the end effector is at least partially submerged comprises instructions to determine that the impedance is above an impedance threshold.

10. The surgical system of claim 9, wherein the one or more characteristics comprises a resonance frequency of the end effector, wherein the instructions to determine that the end effector is at least partially submerged further comprises instructions to determine that the resonance frequency is below a resonance frequency threshold.

11. The surgical system of claim 8, wherein the memory has further instructions to determine a type of liquid in which the end effector is at least partially submerged based on the one or more characteristics, wherein the notification indicates the type of liquid.

12. The surgical system of claim 8, wherein the one or more characteristics comprises an impedance of the end effector, wherein the instructions to determine that the end effector is at least partially submerged comprises instructions to determine that the impedance is above a first threshold, wherein the memory has further instructions to determine that the end effector is in contact with an object while at least partially submerged inside the liquid in response to the impedance being above a second threshold that is greater than the first threshold.

13. The surgical system of claim 12, wherein the notification indicates that the end effector is in contact with an object while submerged in liquid.

14. The surgical system of claim 8, wherein the memory has further instructions to
while the end effector is in air,
determine a temperature of the end effector, and
display the temperature on the display; and
in response to determining that the end effector is at least partially submerged, replace the temperature with an estimated temperature of the liquid.

15. A non-transitory machine-readable medium having instructions which when executed by a processor a surgical system causes the surgical system to
determine one or more characteristics of an end effector of an ultrasonic instrument,
determine that the end effector is at least partially submerged within a liquid based on the determined one or more characteristics, and
in response, display a notification on a display of the surgical system indicating that the end effector is at least partially submerged within the liquid.

16. The non-transitory machine-readable medium of claim 15, wherein the one or more characteristics comprises an impedance of the end effector, wherein the instructions to determine that the end effector is at least partially submerged comprises instructions to determine that the impedance is above an impedance threshold.

17. The non-transitory machine-readable medium of claim 16, wherein the one or more characteristics comprises a resonance frequency of the end effector, wherein the instructions to determine that the end effector is at least partially submerged further comprises instructions to determine that the resonance frequency is below a resonance frequency threshold.

18. The non-transitory machine-readable medium of claim 15 has further instructions to determine a type of liquid in which the end effector is at least partially submerged based on the one or more characteristics, wherein the notification indicates the type of liquid.

19. The non-transitory machine-readable medium of claim 15, wherein the one or more characteristics comprises an impedance of the end effector, wherein the instructions to determine that the end effector is at least partially submerged comprises instructions to determine that the impedance is above a first threshold, wherein the non-transitory machine-readable medium has further instructions to determine that the end effector is in contact with an object while at least partially submerged inside the liquid in response to the impedance being above a second threshold that is greater than the first threshold.

20. The non-transitory machine-readable medium of claim 19, wherein the notification indicates that the end effector is in contact with an object while submerged in liquid.

21. The non-transitory machine-readable medium of claim 15 has further instructions to
while the end effector is in air,
determine a temperature of the end effector, and
display the temperature on the display; and
in response to determining that the end effector is at least partially submerged, replace the temperature with an estimated temperature of the liquid.
